(19) United States
(12) Patent Application Publication
(10) Pub. No.: US 2020/0321090 A1
(43) Pub. Date: Oct. 8, 2020

(54) Portable Electronic Medical Record Card and Associated Systems and Methods

(71) Applicant: Portable EMR Solutions, Inc., East Brunswick, NJ (US)

(72) Inventors: Sherif Sonbol, Princeton, NJ (US); Krishna Reddy Kundur, New York, NY (US)

(73) Assignee: Portable EMR Solutions, Inc., East Brunswick, NJ (US)

(21) Appl. No.: 16/374,149

(22) Filed: Apr. 3, 2019

(51) Int. Cl. G16H 10/65; G16H 15/00

(52) U.S. Cl. CPC G16H 10/65; G06F 21/6245; G16H 15/00

(57) ABSTRACT

A portable electronic medical record card and method for wirelessly communicating electronic medical record information stored on the portable electronic medical record card to a plurality of different information technology systems is provided. The portable electronic medical record card comprises a processor in communication with a memory and biometric and wireless communication modules. The biometric module is configured to receive a biometric input from a user, authenticate the user's identity based on the received biometric input from the user, and authorize the processor to establish wireless communication between the portable electronic medical record card and each of the plurality of different information technology systems based on a result of the authentication. The wireless communication module wirelessly communicates the electronic medical record information stored in the memory to each of the plurality of different information technology systems when the user's identity is authenticated, and receives updated electronic medical information from each of the plurality of different information technology systems.

### BACKGROUND

### Technical Field

The present disclosure relates generally to the field of electronic medical recordkeeping systems. More specifically, the present disclosure relates to a portable electronic medical record card and associated systems and methods.

### Related Art

In the electronic medical record field, increasingly sophisticated systems are being developed to readily access patient medical records. Such systems include, but are not limited to, a smart/proximity card having at least one of an integrated circuit and electrically erasable and programmable memory. These systems can require an administrator (e.g., a medical professional) to write data to the programmable memory of the smart/proximity card via an interface (e.g, a computer) in communication with the system. As such, these systems are disadvantageous because they are static, limited in operating range, lack data processing power, suffer from reduced communication speeds and storage capabilities, and are susceptible to human error during the data writing process.

Therefore, there is currently significant interest in the electronic medical record field in developing a portable electronic medical record card that can securely communicate electronic medical record information stored therein with a plurality of information technology (computing) systems. Further, it would be beneficial to develop a portable electronic medical record card that can automatically update the electronic medical record information stored on the portable electronic medical record card in response to an interaction with a medical professional and/or a visit to a medical facility (e.g., a physician's office and/or hospital) by communicating with an information technology system thereof.

### SUMMARY

The present disclosure relates to a portable electronic medical record card and associated systems and methods. The portable electronic medical record card includes at least one hardware processor configured to establish wireless communication between the portable electronic medical record card and the plurality of disparate information technology systems. The hardware processor is also configured to update the electronic medical record information stored on the portable electronic medical record card in response to an operation performed by at least one of the plurality of information technology systems on electronic medical record information wirelessly communicated.

### BRIEF DESCRIPTION OF THE DRAWINGS

The foregoing features of the present disclosure will be apparent from the following Detailed Description of the Invention, taken in connection with the accompanying drawings, in which:

FIG. 1 is a diagram illustrating the portable electronic medical record card of the present disclosure;

FIG. 2 is a diagram illustrating hardware and software components of the portable electronic medical record card of FIG. 1;

FIG. 3 is a diagram illustrating a plurality of communication links 49a-49f of the present disclosure;

FIG. 4 is a flowchart illustrating processing steps for establishing communication between the portable electronic medical record card of the present disclosure and the plurality of information technology systems;

FIG. 5 is a flowchart illustrating Step 52 of FIG. 4 in greater detail;

FIG. 6 is a flowchart illustrating Step 54 of FIG. 4 in greater detail;

FIG. 7 is a flowchart illustrating Step 56 of FIG. 4 in greater detail;

FIG. 8 is a flowchart illustrating Step 58 of FIG. 4 in greater detail;

FIG. 9 is a flowchart illustrating Step 60 of FIG. 4 in greater detail;

FIG. 10 is a flowchart illustrating Step 62 of FIG. 4 in greater detail; and

FIG. 11 is a diagram illustrating steps for activating the portable electronic medical record card of the present disclosure.

### DETAILED DESCRIPTION

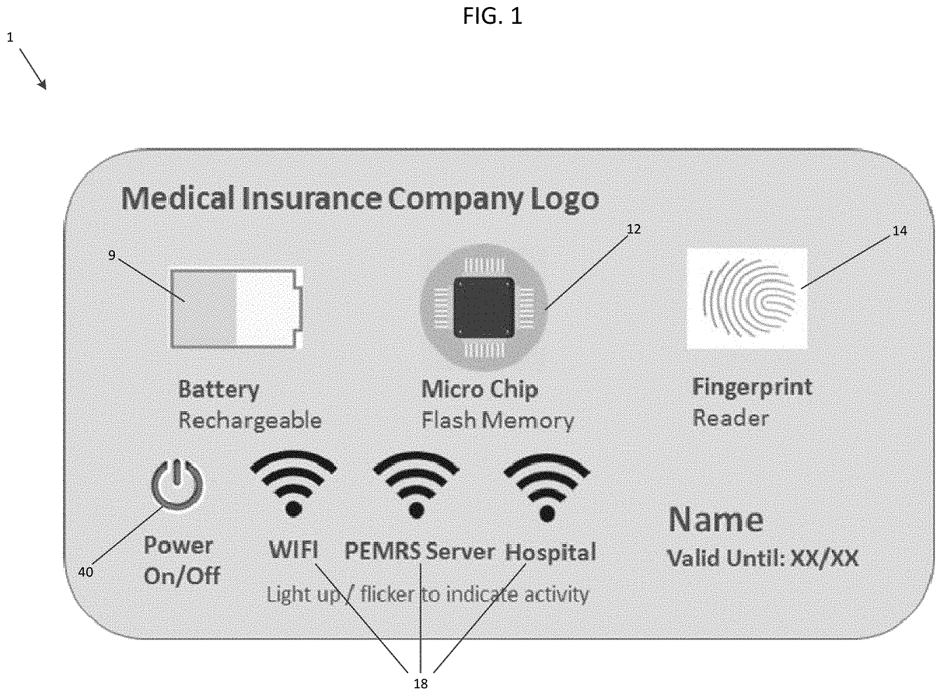

The present disclosure relates to a portable electronic medical record card and method for wirelessly communicating electronic medical record information stored on the portable electronic medical record card to a plurality of information technology systems, as described in detail below in connection with FIGS. 1-11. It should be understood that the electronic medical record card can also receive electronic medical record information from the plurality of information technology systems. It should also be understood that the direct reception of the electronic medical record information by the portable electronic medical record card can be restricted.

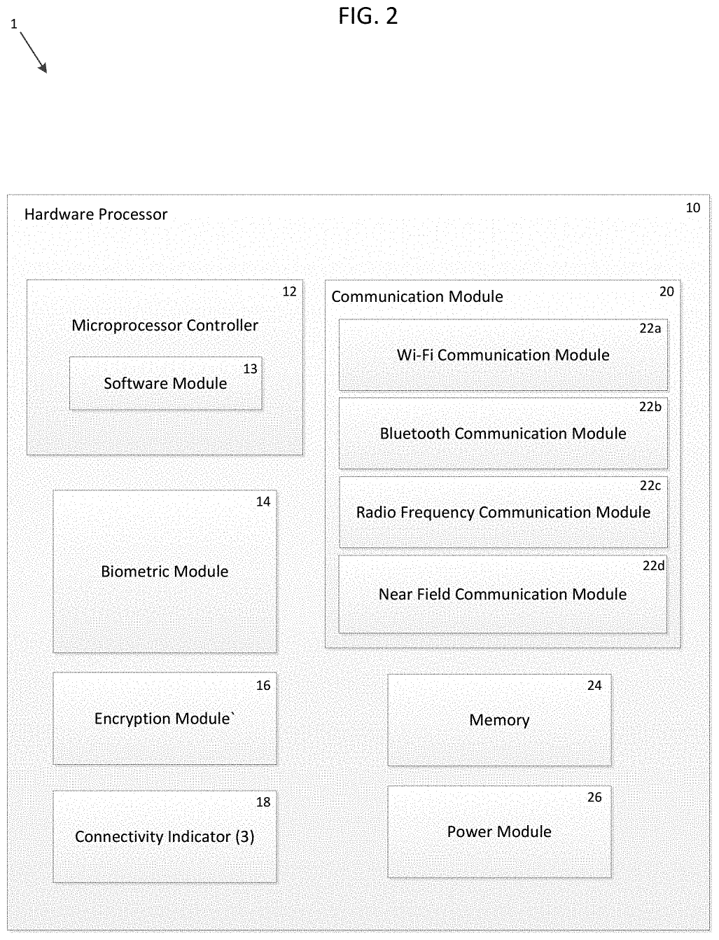

FIGS. 1 and 2 illustrate a portable electronic medical record card 1 of the present disclosure. FIG. 1 is a diagram illustrating the portable electronic medical record card 1. FIG. 2 is a diagram illustrating hardware and software components of the portable electronic medical record card 1 of FIG. 1. A detailed discussion of the card 1 is provided below, with reference to both FIGS. 1 and 2 together.

The portable electronic medical record card 1 could be embodied as a central processing unit (e.g. a hardware processor 10) coupled to various hardware and software components. The hardware processor 10 can include: a controller 12 having a software module 13; a biometric module 14; an encryption module 16; a connectivity indicator 18; a communication module 20 having a Wi-Fi communication module 22a, a Bluetooth™ communication module 22b, a radio frequency communication module 22c and a near field communication (NFC) module 22d; a memory 24; and a power module 26.

The hardware processor 10 could be an Espressif ESP32 processor having at least 2 GB of memory 24 (e.g., flash memory) such that data can be read from and written to the portable electronic medical record card 1. The data can be stored in Javascript Object Notation (JSON) format or Extensible Markup Language (XML) format such that the data is ready for HL7 Fast Healthcare Interoperability Resources (FHIR) standard based communication among various entities and/or parties including, but not limited to, the portable electronic medical record card 1 and the plurality of information technology systems (e.g., a physician's office computing server, a hospital data management system, a cloud-based server system and the Portable Electronic Medical Record Solutions (PEMRS) cloud-based platform). The hardware processor 10 is configured to support the encryption and decryption of stored electronic medical record information via the encryption module 16 having cryptographic hardware acceleration including, but not limited to, Secure Hash Algorithms (SHA), Rivest-Shamir-Adleman (RSA), Advanced Encryption Standard (AES) and a Random Number Generator (RNG). It is noted that the stored electronic medical record information comprises the incomplete or complete past and present medical history of a patient including, but not limited to, prescription history, past surgical history, vaccination history, familial and childhood disease history, allergies, sexual history, drug use, etc.

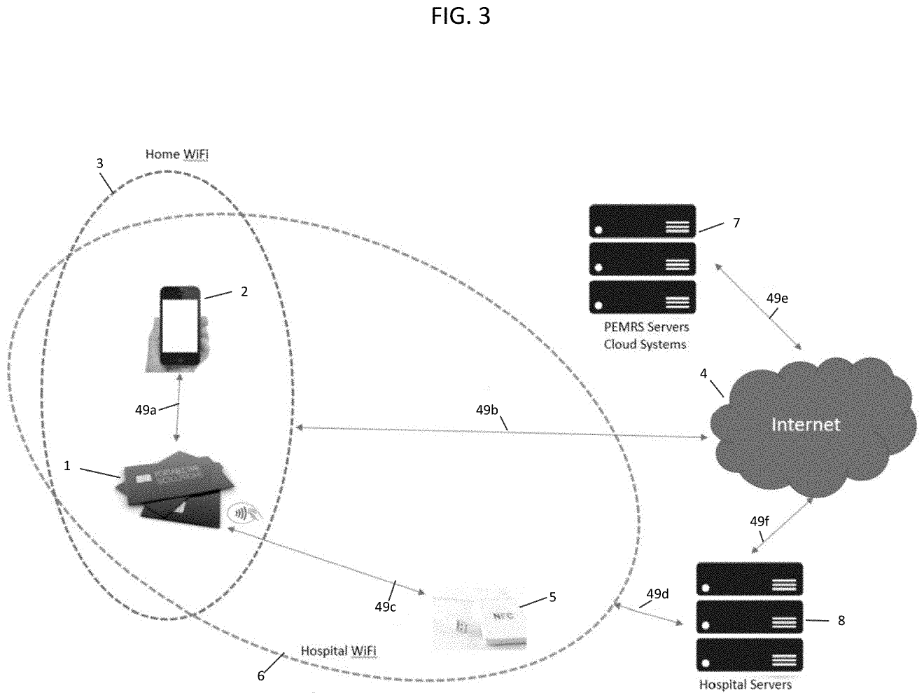

The hardware processor 10 is also configured to support various communication standards and protocols via the communication module 20. For example, the communication module 20 is configured to wirelessly communicate electronic medical record information between the portable electronic medical record card 1 and the plurality of information technology systems through the Wi-Fi communication module 22a, the Bluetooth™ communication module 22b, the radio frequency communication module 22c and the NFC module 22d. It should be understood that the electronic medical record card 1 can also receive electronic medical record information from the plurality of information technology systems. It should also be understood that the direct reception of the electronic medical record information by the portable electronic medical record card 1 can be restricted. For example, the electronic medical record information can be transmitted to an intermediate location (e.g., a Portable Electronic Medical Record Solutions (PEMRS) cloud-based server 7 as shown in FIG. 3) such that the PEMRS cloud-based server 7 can apply appropriate security rules and/or checks before transmitting the electronic medical record information to the portable electronic medical record card 1. The communication module 20 is also configured to support network communication protocols including, but not limited to, TCP/IP and HTTP/HTTPS. The NFC module 22d (e.g., an NFC antenna) is configured to be read by a third party NFC reader (e.g., a medical provider NFC reader) and as such establish a communication link between the portable electronic medical record card 1 and an information technology system to which the third party NFC reader belongs.

The connectivity indicators 18 could comprise a plurality of indicators (e.g., light-emitting diodes (LEDs)) corresponding to a specific entity and/or information technology system to indicate when a communication link between the portable electronic medical record card 1 and the specific entity and/or information technology system is established. For example, an LED indicator could respectively be illuminated orange when the communication link is processing, green when the communication link is established, or red when a problem in the communication link is encountered.

The controller 12 could be a Pycom™ GPy controller configured to run an operating system of the portable electronic medical record card 1. The software module 13 may comprise the logical data model (card file system) and an applet (i.e., a utility program) corresponding to an external system and/or application that can communicate with the portable electronic medical record card 1. The biometric module 14 can be a Pycom™ Pyscan biometric scanner. Specifically, the biometric module 14 could be a fingerprint scanner configured to authenticate an identity of a user of the portable electronic medical record card 1 and grant authorization to establish a communication link between the portable electronic medical record card 1 and an entity and/or information technology system. The biometric module 14 could comprise the NFC module 22d (e.g., an NFC antenna). The power module 26 could be a rechargeable lithium ion battery. The power module 26 could have ON/OFF capability such that the portable electronic medical record card 1 could be powered ON when necessary and turned OFF when not in use to prolong battery life.

As shown in FIG. 1, several of the above described components of the portable electronic medical record card 1 can be visible on a surface thereof including, but not limited to, the battery life indicator 9, the hardware processor 12, the biometric module 14, the connectivity indicator 18, and a power ON/OFF button 40. The power ON/OFF button 40 is configured to power the portable electronic medical record card 1 ON or to turn the portable electronic medical record card 1 OFF when not in use to prolong battery life. The portable electronic medical record card 1 can transition from ON to a Sleep Mode and from the Sleep Mode to OFF. The battery life indicator 9 displays an estimated remaining battery life of the portable electronic medical record card 1. The battery life indicator 9 can display the estimated remaining battery life as a graphic image and/or a percentage. As discussed, the biometric module 14 could be a fingerprint scanner configured to authenticate an identity of a user of the portable electronic medical record card 1 and grant authorization to establish a communication link between the portable electronic medical record card 1 and an entity and/or information technology system. The connectivity indicators 18 comprise a plurality of LED indicators including a Wi-Fi connectivity indicator, a PEMRS cloud server connectivity indicator, and a hospital server connectivity indicator, for example. Each of the connectivity indicators 18 may indicate that a communication link has been established between the portable electronic medical record card 1 and the entity and/or information technology system when the respective indicator 18 is illuminated. In addition, the Wi-Fi indicator 18 may also be configured to turn the Wi-Fi communication module 22a of the portable electronic medical record card 1 ON or to turn the Wi-Fi communication module 22a OFF when pressed by a user. The Wi-Fi communication module 22a may unilaterally enter a sleep mode when not in use such that the Wi-Fi indicator 18 may cease to be illuminated.

FIG. 3 is a diagram illustrating a plurality of communication links 49a-49f of the present disclosure. Components of the communication links include, but are not limited to, the portable electronic medical record card 1, a remote device 2, a home Wi-Fi network 3, the internet 4, a NFC reader 5, a hospital Wi-Fi network 6, a Portable Electronic Medical Record Solutions (PEMRS) cloud-based server 7 and a hospital server 8. It should be noted that the term “hospital” is not limited to a hospital, but can also include a medical provider generally (e.g., an urgent care center, a physician's office, an ambulance, a triage center, an emergency medical technician as part of an emergency response, etc.). The portable electronic medical record card 1 can wirelessly communicate electronic medical record information stored on the portable electronic medical record card 1 to the plurality of information technology systems shown in FIG. 3. It should be understood that the electronic medical record card 1 can also receive electronic medical record information from the plurality of information technology systems. It should also be understood that the direct reception of the electronic medical record information by the portable electronic medical record card 1 can be restricted. For example, the electronic medical record information can be transmitted to the PEMRS cloud-based server 7 such that the PEMRS cloud-based server 7 can apply appropriate security rules and/or checks before transmitting the electronic medical record information to the portable electronic medical record card 1.

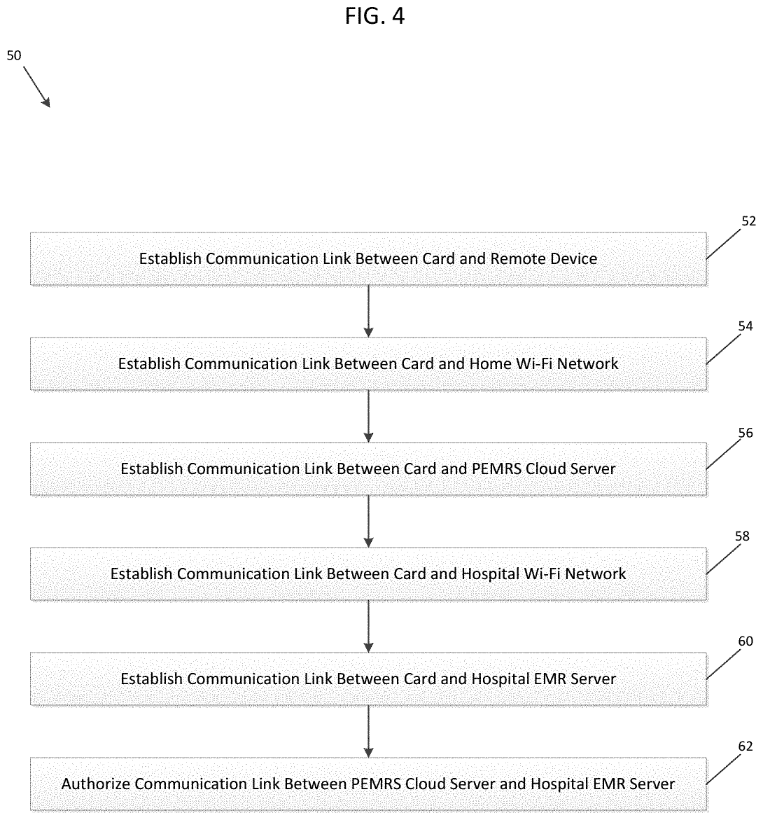

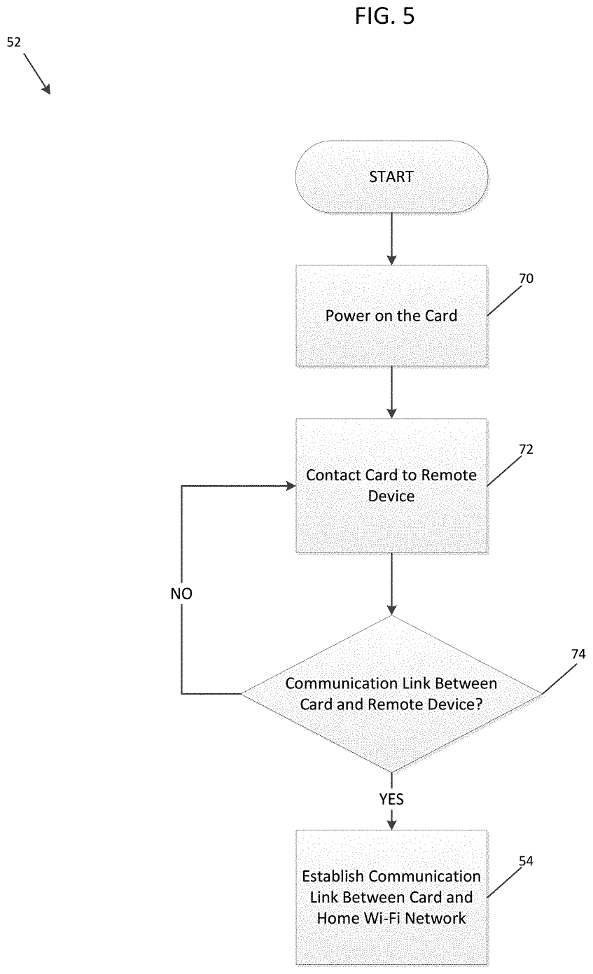

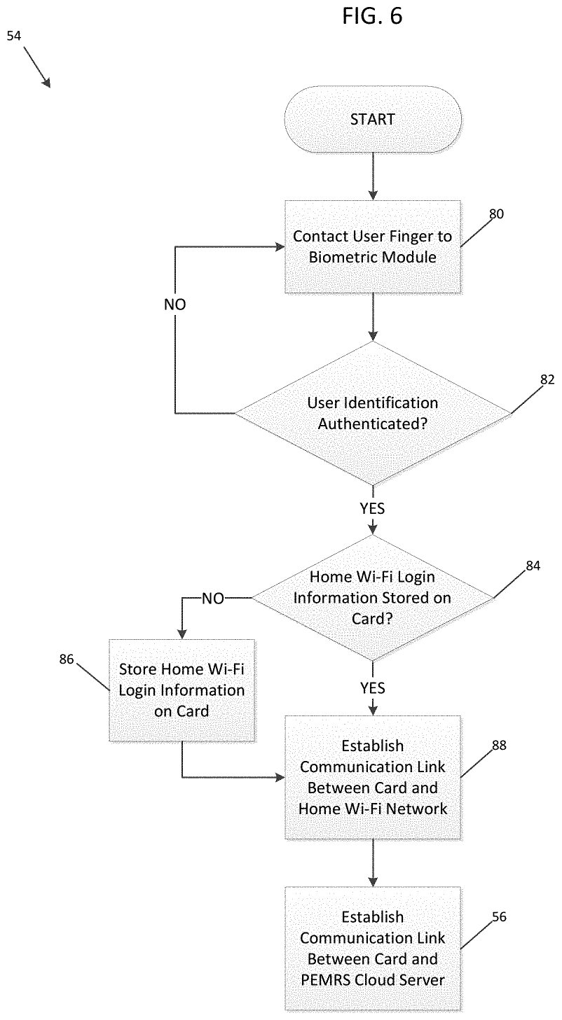

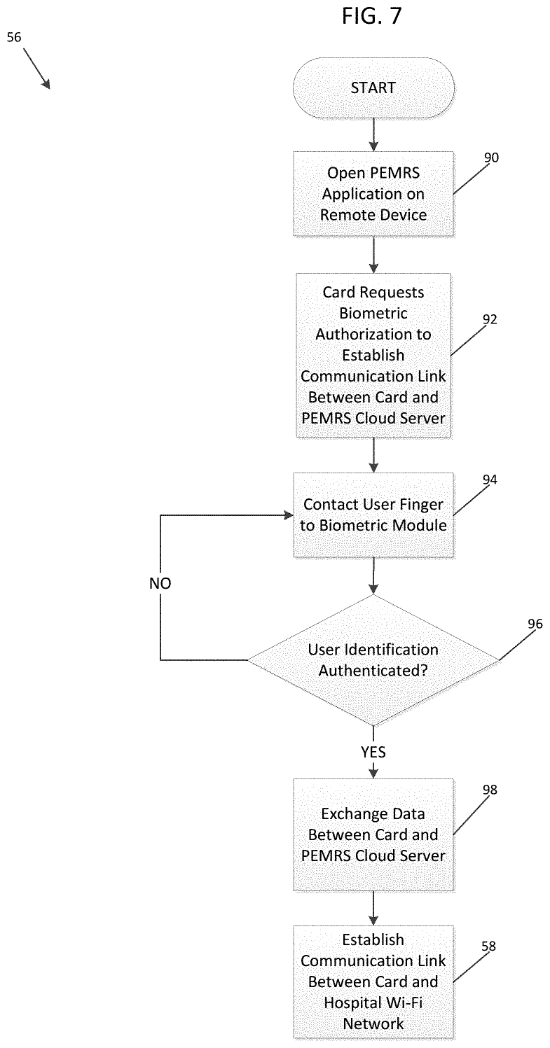

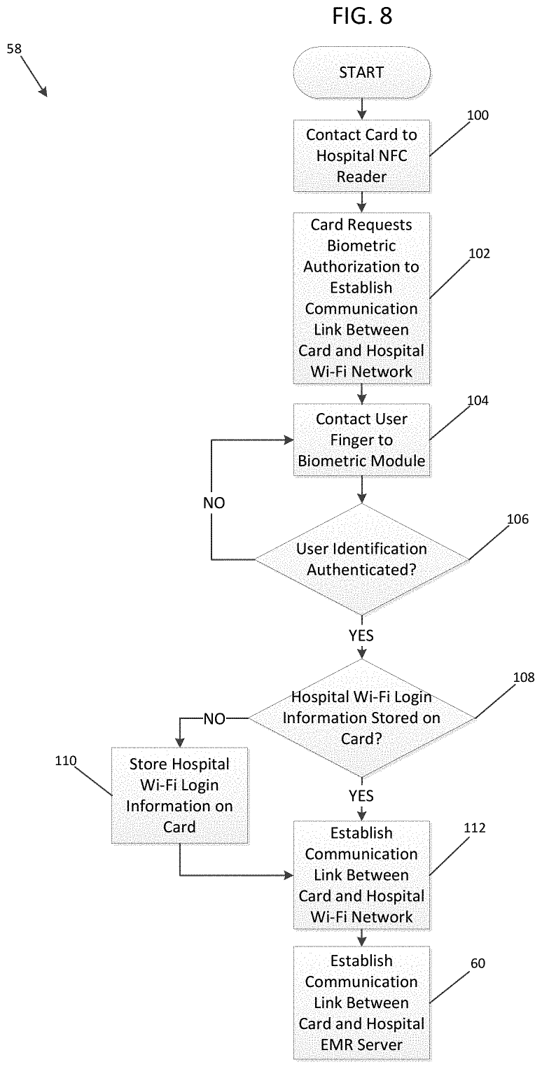

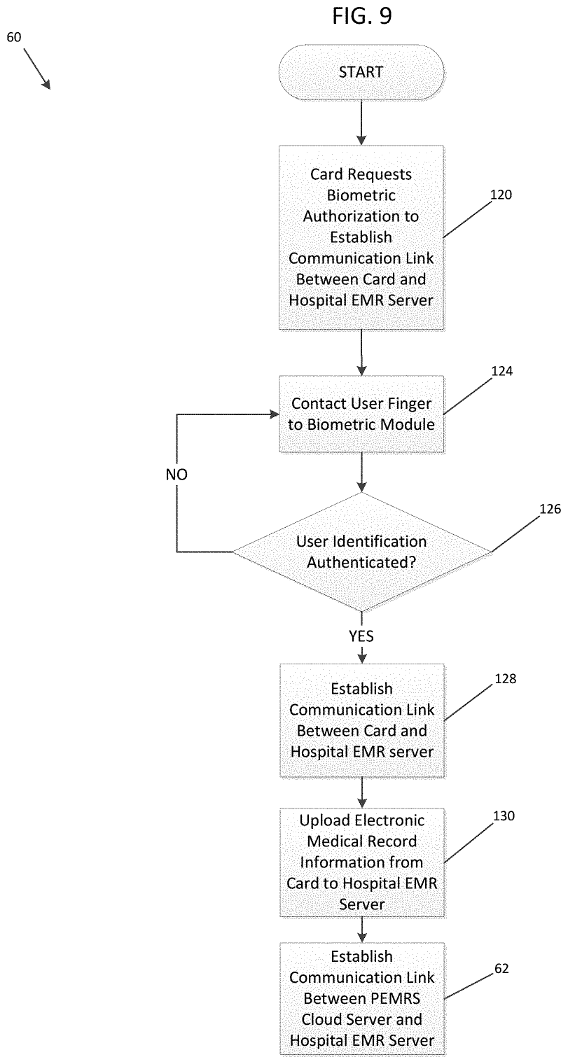

FIG. 4 is a flowchart illustrating processing steps 50 carried out by the portable electronic medical record card 1 of the present disclosure to establish the communication links 49a-49f with the plurality of information technology systems 3-8. In step 52, the portable electronic medical record card 1 establishes a communication link 52a with the remote device 2, which is discussed in greater detail in connection with FIG. 5. The remote device 2 may include, but is not limited to, a cellular telephone, a tablet, a personal digital assistant (PDA), etc. In step 54, the portable electronic medical record card 1 establishes a communication link with a home Wi-Fi network 3 of a user of the portable electronic medical record card 1, which is discussed in greater detail in connection with FIG. 6. In step 56, the portable electronic medical record card 1 establishes a communication link 49e with at least one of the plurality of PEMRS cloud based servers 7 which can be a part of a PEMRS information technology system, via the Internet 4, which is discussed in greater detail in connection with FIG. 7. The method then proceeds to step 58 wherein the portable electronic medical record card 1 establishes a communication link 49c with a hospital Wi-Fi network 6 via the hospital NFC reader 5, which is discussed in greater detail in connection with FIG. 8. In step 60, the portable electronic medical record card 1 establishes a communication link 49d with at least one of a plurality of hospital servers 8 of a hospital information technology system via the hospital Wi-Fi network 6, which is discussed in greater detail in connection with FIG. 9. In step 62, the portable electronic medical record card 1 authorizes a communication link 49f between the PEMRS information technology system (e.g., the PEMRS cloud-based server 7) and the hospital information technology system (e.g., the hospital servers 8) via the Internet 4 such that user's electronic medical record information can be exchanged between at least one of the plurality of PEMRS cloud servers 7 and at least one of the plurality of hospital servers 8. It should be noted that one or more of steps 56, 58 and 60 must be established to proceed to step 62. It should also be understood that the above-described communication links need not be established at the same time or in the forgoing order. For example, communication links 49b and 49c can be established independent of one another.

FIG. 5 is a flowchart illustrating Step 52 of FIG. 4 in greater detail. Beginning in step 70, a user powers on the portable electronic medical record card 1 by pressing the power ON/OFF button 40 (see FIG. 1). Particularly, as shown in FIG. 1, the portable electronic medical record card 1 does not include a keypad or mechanism to input information (e.g., a user name, a Wi-Fi network name, a Wi-Fi password, medical record information, etc.) that can be used by, or stored in the memory 24 of the portable electronic medical record card 1. In step 72 the user contacts the portable electronic medical record card 1 to the remote device 2 to establish a communication link between the portable electronic medical record card 1 and the remote device 2 via NFC. As such, the portable electronic medical record card 1 is linked to the remote device 2 which the user can use to input information to be stored in the memory 24 of the portable electronic medical record card 1 (e.g., via an application of the remote device 2). In step 74, the portable electronic medical record card 1 determines if a communication link has been successfully established with the remote device 2. If the communication link is not successfully established then the process returns to step 72 and the user may contact the portable electronic medical record card 1 to the remote device 2 again to re-attempt to establish a communication link between the portable electronic medical record card 1 and the remote device 2. If the communication link is successful, then the process proceeds to step 54 to establish a communication link between the portable electronic medical record card 1 and the home Wi-Fi network 3.

FIG. 6 is a flowchart illustrating Step 54 of FIG. 4 in greater detail. Beginning in step 80, the user can scan his/her finger on the biometric module 14 (see FIG. 1). In step 82, the portable electronic medical record card 1 determines whether an identification of the user is authenticated. Specifically, the biometric module 14 captures the scanned fingerprint image, compresses the image with a Wavelet Scalar Quantization (WSQ) algorithm, transforms the image into a standard template (e.g., ISO 1974-2 or ANSI 378), compares the captured scanned fingerprint with a verified fingerprint of the user stored on the portable electronic medical record card 1, and returns a pass or fail authentication status based on the comparison. If the biometric module 14 does not authenticate the identity of the user, then the process returns to step 80 and the user can scan his/her finger on the biometric module 14 again.

If the identity of the user is authenticated, then the process proceeds to step 84 where it is determined whether the home Wi-Fi network 3 login information (e.g., network name and network password) is stored on the portable electronic medical record card 1. If the home Wi-Fi network 3 login information is not stored on the portable electronic medical record card 1 then the process proceeds to step 86 where the portable electronic medical record card 1 receives the home Wi-Fi network 3 login information from the remote device 2, and stores the home Wi-Fi network 3 information on the portable electronic medical record card 1. If the home Wi-Fi network 3 login information is stored on the portable electronic medical record card 1 the process proceeds to step 88 where the process establishes a communication link between the portable electronic medical record card 1 and the home Wi-Fi network 3 by logging into the home Wi-Fi network 3. Once the communication link is established between the portable electronic medical record card 1 and the home Wi-Fi network 3, the process 54 proceeds to step 56 to establish the communication link 49e between the portable electronic medical record card 1 and the PEMRS cloud-based server 7.

FIG. 7 is a flowchart illustrating Step 56 of FIG. 4 in greater detail. Beginning in step 90, the user may open the PEMRS application on the remote device 2, which can be used to backup or restore electronic medical record information from the portable electronic medical record card 1 to the PEMRS cloud-based server 7. In addition, the PEMRS application can perform create, read, update, delete (CRUD) operations on the electronic medical record information stored on the portable electronic medical record card 1. Specifically, middleware software is used to perform the CRUD operations wherein access to the memory 24 is via the middleware software operating on the portable electronic medical record card 1.

In step 92, the portable electronic medical record card 1 requests biometric authorization to establish the communication link 49e between the portable electronic medical record card 1 and the PEMRS cloud-based server 7. In step 94, the user scans his/her finger on the biometric module 14 (see FIG. 1) to authenticate his/her identity and thereby grant authorization to establish the communication link 49e between the portable electronic medical record card 1 and the PEMRS cloud-based server 7. As detailed above, the biometric module 14 captures and compares the scanned fingerprint image with a verified fingerprint of the user stored on the portable electronic medical record card 1 and returns a pass or fail authentication status based on the comparison. In step 96, the process 56 determines whether the identification of the user is authenticated. If the biometric module 14 does not authenticate the identity of the user, then the process returns to step 94 and the user can scan his/her finger on the biometric module 14 again. If the user's identity is authenticated then the process proceeds to step 98.

In step 98, data including, but not limited to, electronic medical record information is exchanged between the portable electronic medical record card 1 and the PEMRS cloud-based server 7. The portable electronic medical record card 1 has a unique identifier and token (i.e., password) and the PEMRS cloud-based server 7 has a Uniform Resource Locator (URL). The combination of the unique identifier and token, and the URL allows for both the portable electronic medical record card 1 and the PEMRS cloud-based server 7 to be authenticated thus creating a secure data exchange between the portable electronic medical record card 1 and the PEMRS cloud-based server 7. The portable electronic medical record card 1 and the PEMRS cloud-based server 7 can communicate via HTTPS and HTTP. If the user is in the vicinity of the hospital Wi-Fi network 6, the process proceeds to step 58 to establish a communication link between the portable electronic medical record card 1 and the hospital Wi-Fi network 6.

FIG. 8 is a flowchart illustrating Step 58 of FIG. 4 in greater detail. Beginning in step 100, a user may place the portable electronic medical record card 1 in close proximity to or in contact with a hospital NFC reader 5 to allow the hospital NFC reader 5 to identify the portable electronic medical record card 1. In step 102, the portable electronic medical record card 1 requests biometric authorization to establish the communication link between the portable electronic medical record card 1 and the hospital Wi-Fi network 6. In step 104, the user scans his/her finger on the biometric module 14 (see FIG. 1) to authenticate his/her identity and thereby grant authorization to establish the communication link between the portable electronic medical record card 1 and the hospital Wi-Fi network 6. As detailed above, the biometric module 14 captures and compares the scanned fingerprint image with a verified fingerprint image of the user stored on the portable electronic medical record card 1 and returns a pass or fail authentication status based on the comparison. In step 106, the process 58 determines whether the identification of the user is authenticated. If the biometric module 14 does not authenticate the identity of the user, then the process returns to step 104 and the user can scan his/her finger on the biometric module 14 again.

If the identity of the user is authenticated, then the process proceeds to step 108 where it is determined whether the hospital Wi-Fi network 6 login information (e.g., network name and network password) is stored on the portable electronic medical record card 1. If the hospital Wi-Fi network 6 login information is not stored on the portable electronic medical record card 1 then the process proceeds to step 110 where the portable electronic medical record card 1 receives the hospital Wi-Fi network 6 login information from the remote device 2, and stores the hospital Wi-Fi network 6 information on the portable electronic medical record card 1. If the hospital Wi-Fi network 6 login information is stored on the portable electronic medical record card 1 the process proceeds to step 112 where the process 58 establishes a communication link between the portable electronic medical record card 1 and the hospital Wi-Fi network 6 by logging into the hospital Wi-Fi network 6. Once the communication link is established between the portable electronic medical record card 1 and the hospital Wi-Fi network 6, the process 58 proceeds to step 60 to establish the communication link 49d between the portable electronic medical record card 1 and the hospital EMR server 8.

It is noted that in emergency situations it may be difficult to establish a communication link between the portable electronic medical record card 1 and a medical professional (e.g., a paramedic) providing life saving emergency care when a user of the portable electronic medical record card 1 is incapacitated and/or Wi-Fi is unavailable. In such a situation, a communication link between the portable electronic medical record card 1 and the medical professional or an ambulance system can be established via NFC or Bluetooth™ communication in combination with biometric authorization.

FIG. 9 is a flowchart illustrating Step 60 of FIG. 4 in greater detail. Beginning in step 120, the portable electronic medical record card 1 requests biometric authorization to establish the communication link 49d between the portable electronic medical record card 1 and the hospital EMR server 8. In step 124 the user scans his/her finger on the biometric module 14 (see FIG. 1) to authenticate his/her identity and thereby grant authorization to establish the communication link 49d between the portable electronic medical record card 1 and the hospital EMR server 8. As detailed above, the biometric module 14 captures and compares the scanned fingerprint image with a verified fingerprint image of a user stored on the portable electronic medical record card 1 and returns a pass or fail authentication status based on the comparison. In step 126, the process 60 determines whether an identification of the user is authenticated. If the biometric module 14 does not authenticate the identity of the user, then the process returns to step 124 and the user can scan his/her finger on the biometric module 14 again. If the identity of the user is authenticated, then the process 60 proceeds to step 128 where the process 60 establishes the communication link 49d between the portable electronic medical record card 1 and the hospital EMR server 8. Then in step 130, the portable electronic medical record card 1 uploads the stored electronic medical record information to the hospital EMR server 8. The process 60 then proceeds to step 62 to establish the communication link 49f between the PEMRS cloud-based server 7 and the hospital EMR server 8.

Communication between the portable electronic medical record card 1 and the hospital EMR server 8 can be supported by HTTP and HTTPS wherein FHIR provides an application interface for managing the electronic health record. For example, FHIR provides a representational state transfer application program interface (REST API) having a rich but simple set of interactions including, but not limited to:
Create=POST https://example.com/path/{resourceType};
Read=GET https://example.com/path/{resourceType}/{id};
Update=PUT https://example.com/path/{resourceType}/{id};
Delete=DELETE https://example.com/path/{resourceType}/{id};
Search=GET https://example.com/path/{resourceType}?search parameters . . . ;
History=GET https://example.com/path/{resourceType}/{id}/_history;
Transaction=POST https://example.com/path/(POST a transaction bundle to the system); and
Operation=GET https://example.com/path/{resourceType}/{id}/${opname}.

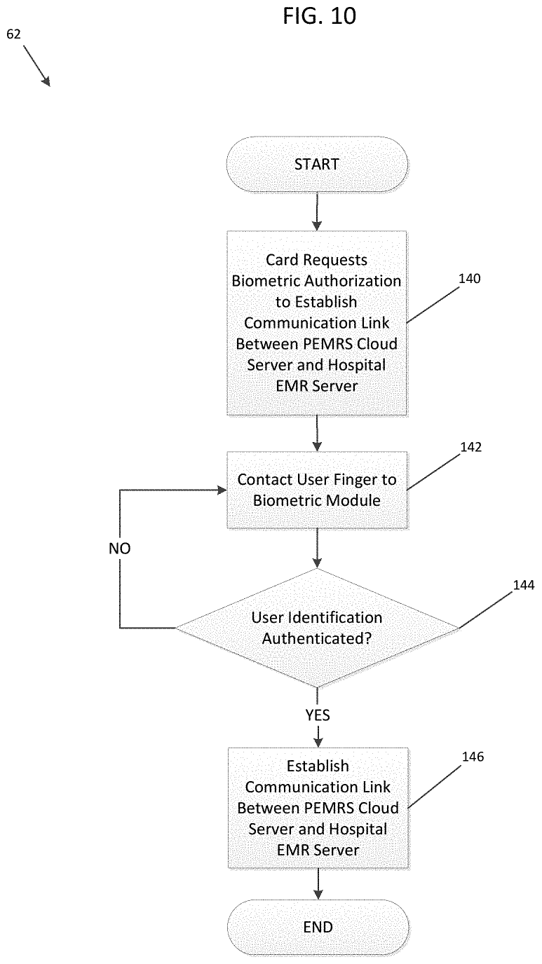

FIG. 10 is a flowchart illustrating Step 62 of FIG. 4 in greater detail. Beginning in step 140, the portable electronic medical record card 1 requests biometric authorization to establish a communication link 49f between the PEMRS cloud-based server 7 and the hospital EMR server 8. In step 142, the user scans his/her finger on the biometric module 14 (see FIG. 1) to authenticate his/her identity and thereby grant authorization to establish the communication link between the PEMRS cloud-based server 7 and the hospital EMR server 8. As detailed above, the biometric module 14 captures and compares the scanned fingerprint image with a verified fingerprint image of the user stored on the portable electronic medical record card 1 and returns a pass or fail authentication status based on the comparison. In step 144, the process 62 determines whether an identification of the user is authenticated. If the biometric module 14 does not authenticate the identity of the user, then the process returns to step 142 and the user can scan his/her finger on the biometric module 14 again. If the identity of the user is authenticated, then the process 62 proceeds to step 146 where the process 62 establishes the communication link 49f between the PEMRS cloud-based server 7 and the hospital EMR server 8.

Communication between the PEMRS cloud server 7 and the hospital EMR server 8 can be supported by HTTP and HTTPS. It is noted that the PEMRS cloud-based server 7 and the hospital EMR server 8 may need to communicate directly when electronic medical record information stored on the portable electronic medical record card 1 is incomplete and/or if a medical provider wishes to execute a comprehensive and/or specific search of the user's electronic medical record information.

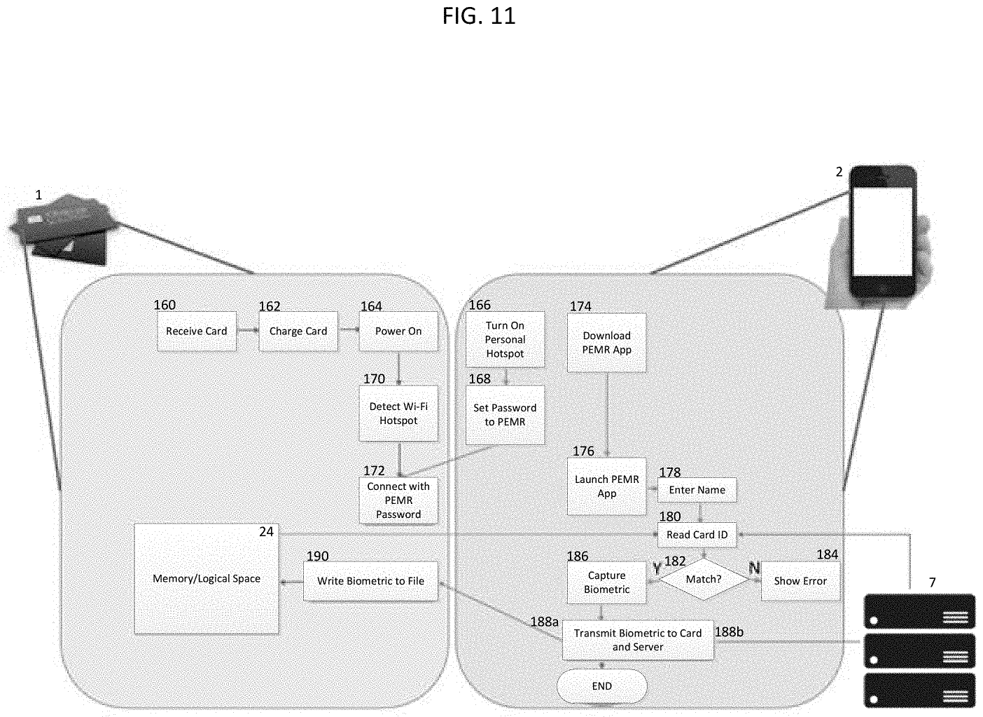

FIG. 11 is a diagram illustrating the steps carried out to activate the portable electronic medical record card 1 of the present disclosure. Specifically, the diagram illustrates steps carried out by each of the portable electronic medical record card 1, the remote device 2, and the PEMRS cloud-based server 7 to activate the portable electronic medical record card 1 over the Internet 4. As shown in FIG. 11, in step 160 a user receives the portable electronic medical record card 1 and subsequently charges the portable electronic medical record card 1 in step 162 before powering the portable electronic medical record card 1 ON in step 164. In step 166, the user turns on a Wi-Fi personal hotspot from the remote device 2, and in step 168 sets the password of the Wi-Fi personal hotspot to “PEMR.” In step 170, the portable electronic medical record card 1 detects the Wi-Fi personal hotspot. It is noted that step 170 can also involve the user pressing the Wi-Fi button and turning the Wi-Fi ON. In step 172, the portable electronic medical record card 1 establishes a connection with the remote device 2. It is noted that the user can change the default password “PEMR” after completing the activation process.

As discussed above, the PEMR Application allows the user to input information to be stored in the memory 24 of the portable electronic medical record card 1 since the portable electronic medical record card 1 does not include a keypad or mechanism to input information. It is noted that the portable electronic medical record card 1 can connect to the remote device 2 using DIAL protocol. In step 174, the user downloads the PEMRS Application to the remote device 2 and launches the PEMR Application in step 176.

In step 178, the user enters his/her user information (e.g., user name) via the PEMR Application on the remote device 2. It is noted that a Globally Unique Identifier (GUID) is stored in the memory 24 of the portable electronic medical record card 1 at the time of shipping. In addition, the portable electronic medical record card 1 GUID is also stored on the PEMRS cloud-based server 7 at the time of shipping. The user name and the GUID are used during the activation process to authenticate the portable electronic medical record card 1. As such, in step 180, the remote device 2 receives the GUID from the memory 24 of the portable electronic medical record card 1 and the GUID from the PEMRS cloud-based server 7 and in step 182 determines whether the respective GUIDs match. If the GUIDs do not match, then the process proceeds to step 184 where the activation process displays an error message on the remote device 2. If the GUIDs match then the process proceeds to step 186 to capture biometric information from the user. It is noted that an indicator on the portable electronic medical record card 1 can blink to indicate a connection to a cloud account.

In step 186, the user's biometric information (e.g., a fingerprint) is captured. For example, the fingerprint of the user can be captured by the PEMR application on the remote device 2. Specifically, the PEMR application captures the scanned fingerprint image, compresses the image with a Wavelet Scalar Quantization (WSQ) algorithm, and transforms the image into a standard template (e.g., ISO 1974-2 or ANSI 378). It is noted that multiple fingerprint samples, e.g., three, may be captured wherein the best sample is compressed and transformed into the standard template. If the fingerprint scan is accepted, the light on the portable electronic medical record card 1 is illuminated a solid green. In steps 188a and 188b, the fingerprint template is transmitted to the portable electronic medical record card 1 and the PEMRS cloud-based server 7 for storage. Finally, in step 190, the portable electronic medical record card 1 writes the transmitted fingerprint template to the memory 24. Upon completion of the activation process, the portable electronic medical record card 1 can wirelessly communicate electronic medical record information stored therein to a plurality of information technology systems. It is noted that the Wi-Fi can enter a sleep state after an absence of use (e.g., three minutes). The portable electronic medical record card 1 can be activated and used in various situations including, but not limited to, initial activation, check-in and check out during a visit with a medical provider, reviewing personal health records via a website, changing insurance providers and in the event the portable electronic medical record card 1 is lost or stolen.

Having thus described the present disclosure in detail, it is to be understood that the foregoing description is not intended to limit the spirit or scope thereof. What is desired to be protected is set forth in the following claims.

1. A portable electronic medical record card for wirelessly communicating electronic medical record information stored on the portable electronic medical record card to a plurality of information technology systems, comprising:
a memory storing the electronic medical record information;
a biometric module configured to receive a biometric input from a user;
a wireless communication module; and
a processor in communication with the memory, the biometric module, and the wireless communication module, the processor configured to establish wireless communication between the portable electronic medical record card and the plurality of different information technology systems,
wherein the biometric module:
receives the biometric input from the user,
authenticates the user's identity based on the received biometric input from the user, and
authorizes the processor to establish wireless communication between the portable electronic medical record card and each of the plurality of information technology systems based on a result of the authentication, and
wherein the wireless communication module:
wirelessly communicates the electronic medical record information stored in the memory to each of the plurality of different information technology systems when the user's identity is authenticated, and
receives updated electronic medical information from each of the plurality of different information technology systems.

2. The portable electronic medical record card of claim 1, wherein the plurality of information technology systems include a Wi-Fi network, a medical provider data system, and a cloud-based electronic medical record server system.

3. The portable electronic medical record card of claim 1, wherein the biometric module is a fingerprint reader and authenticates the user's identity by capturing and comparing a scanned fingerprint image of the user with a verified fingerprint image of the user.

4. The portable electronic medical record card of claim 1, wherein the wireless communication module is configured to communicate the electronic medical record information stored in the memory to the plurality of information technology systems via Wi-Fi, Near Field Communication, Bluetooth™ or radio frequency.

5. The portable electronic medical record card of claim 1, further comprising an encryption module configured to encrypt and decrypt the electronic medical record information stored in the memory via at least one of Secure Hash Algorithms (SHA), Rivest-Shamir-Adleman (RSA), Advanced Encryption Standard (AES), and a Random Number Generator (RNG).

6. The portable electronic medical record card of claim 1, further comprising:
a power module; and
a power button, wherein
the power module is a rechargeable battery and the power button provides ON/OFF capability.

7. The portable electronic medical record card of claim 1, wherein
the memory is programmable, and
the processor updates the electronic medical record information stored in the memory in response to the wireless communication module receiving the updated electronic medical record information from any of the plurality of different information technology systems.

8. The portable electronic medical record card of claim 1, further comprising at least one connectivity indicator configured to indicate a status of a wireless communication link between the portable electronic medical record card and at least one of the plurality of information technology systems.

9. A method for wirelessly communicating electronic medical record information stored on a portable electronic medical record card to a plurality of different information technology systems, comprising:
establishing wireless communication between the portable electronic medical record card and a first information technology system among the plurality of different information technology systems by
receiving a first biometric input from a user,
authenticating the user's identity based on the received first biometric input from the user,
authorizing the processor to establish wireless communication between the portable electronic medical record card and the first information technology system among the plurality of different information technology systems based on a result of the authenticating,
wirelessly communicating the electronic medical record information stored on the portable electronic medical record card to the first information technology system among the plurality of different information technology systems when the user's identity is authenticated, and
receiving updated electronic medical information from the first information technology system among the plurality of different information technology system;
establishing wireless communication between the portable electronic medical record card and a second information technology system among the plurality of different information technology systems by
receiving a second biometric input from the user,
authenticating the user's identity based on the received second biometric input from the user,
authorizing the processor to establish wireless communication between the portable electronic medical record card and the second information technology system among the plurality of different information technology systems based on a result of the authenticating,
wirelessly communicating the electronic medical record information stored on the portable electronic medical record card to the second information technology system among the plurality of different information technology systems when the user's identity is authenticated, and
receiving updated electronic medical information from the second information technology system among the plurality of different information technology system; and
establishing wireless communication between the portable electronic medical record card and a third information technology system among the plurality of different information technology systems by
receiving a third biometric input from the user,
authenticating the user's identity based on the received third biometric input from the user,
authorizing the processor to establish wireless communication between the portable electronic medical record card and the third information technology system among the plurality of different information technology systems based on a result of the authenticating,
wirelessly communicating the electronic medical record information stored on the portable electronic medical record card to the third information technology system among the plurality of different information technology systems when the user's identity is authenticated, and
receiving updated electronic medical information from the third information technology system among the plurality of different information technology system.

10. The method of claim 9, further comprising encrypting and decrypting the electronic medical record information stored on the portable electronic medical record card via at least one of Secure Hash Algorithms (SHA), Rivest-Shamir-Adleman (RSA), Advanced Encryption Standard (AES), and a Random Number Generator (RNG).

11. The method of claim 9, further comprising indicating a status of a wireless communication link between the portable electronic medical record card and at least one of the first information technology system, the second information technology system and the third information technology system among the plurality of different information technology systems wherein the first information technology system, the second information technology system and the third information technology system include a Wi-Fi network, a medical provider data system and a cloud-based electronic medical record server system.

12. A system for wirelessly communicating electronic medical record information, comprising:
a first information technology system; and
a portable electronic medical record card, the portable electronic medical record card comprising
a memory storing the electronic medical record information;
a biometric module configured to receive a biometric input from a user;
a wireless communication module; and
a processor in communication with the memory, the biometric module, and the wireless communication module, the processor configured to establish wireless communication between the portable electronic medical record card and the first information technology system and a plurality of second information technology systems,
wherein the biometric module:
receives the biometric input from the user,
authenticates the user's identity based on the received biometric input from the user, and
authorizes the processor to establish wireless communication between the portable electronic medical record card and the first information technology system and the plurality of second information technology systems based on a result of the authentication, and
wherein the wireless communication module:
wirelessly communicates the electronic medical record information stored in the memory to the first information technology system and the plurality of second information technology systems when the user's identity is authenticated, and
receives updated electronic medical information from each of the first information technology system and the plurality of second information technology systems.

13. The system of claim 1, wherein the processor updates the electronic medical record information stored in the memory in response to the wireless communication module receiving the updated electronic medical record information from the first information technology system or any of the plurality of second information technology systems.

14. The system of claim 12, wherein the first information technology system and the plurality of second information technology systems include a Wi-Fi network, a medical provider data system and a cloud-based electronic medical record server system.

15. The system claim 12, wherein the biometric module is a fingerprint reader and authenticates the user's identity by capturing and comparing a scanned fingerprint image of the user with a verified fingerprint image of the user.

16. The system of claim 12, wherein the wireless communication module is configured to communicate the electronic medical record information stored in the memory to the plurality of information technology systems via Wi-Fi, Near Field Communication, Bluetooth™ or radio frequency.

17. The system of claim 12, wherein the portable electronic medical record card further comprises an encryption module configured to encrypt and decrypt the electronic medical record information stored in the memory via at least one of Secure Hash Algorithms (SHA), Rivest-Shamir-Adleman (RSA), Advanced Encryption Standard (AES), and a Random Number Generator (RNG).

18. The system of claim 12, wherein the portable electronic medical record card further comprises a power module and a power button, the power module being a rechargeable battery and the power button configured to provide ON/OFF capability.

19. The system of 12, wherein the memory is programmable.

20. The system of claim 12, wherein the portable electronic medical record card further comprises at least one connectivity indicator configured to indicate a status of a wireless communication link between the portable electronic medical record card and at least one of the first technology information system and the plurality of second information technology systems.
